(19) United States
(12) Patent Application Publication
(10) Pub. No.: US 2024/0139535 A1
(43) Pub. Date: May 2, 2024

(54) METHODS AND SYSTEMS FOR SHAPING MAGNETICS FIELDS TO ALIGN FORCES ON MAGNETIC PARTICLES FOR PATIENT ANATOMY AND MOTION

(71) Applicant: Otomagnetics, Inc., Gaithersburg, MD (US)

(72) Inventor: Benjamin Shapiro, Washington, DC (US)

(21) Appl. No.: 18/386,765

(22) Filed: Nov. 3, 2023

Related U.S. Application Data
(63) Continuation of application No. PCT/US2022/072080, filed on May 3, 2022.
(60) Provisional application No. 63/184,072, filed on May 4, 2021.

(51) Int. Cl. A61N 2/02; A61G 12/00; A61G 13/12

(52) U.S. Cl. CPC A61N 2/02; A61G 12/001; A61G 13/121; A61G 2200/14

(57) ABSTRACT

A system and method for determining magnetization and force directions for a plurality of magnetic elements that makes up a magnetic assembly on a medical cart. The magnetic assembly may be configured to generate magnetic forces that create pushing and/or pulling force on a magnetic therapeutic agent at specific locations relative to a simulated patient on the medical cart.

### CROSS-REFERENCE TO RELATED APPLICATIONS

This application claims the benefit under 35 U.S.C. § 119(e) to U.S. Provisional Patent Application No. 63/184,072, titled METHODS AND SYSTEMS FOR SHAPING MAGNETICS FIELDS TO ALIGN FORCES ON MAGNETIC PARTICLES FOR PATIENT ANATOMY AND MOTION, filed May 4, 2021, the disclosure of each of which is herein incorporated by reference in its entirety.

### BACKGROUND

This application relates to magnetic systems for delivery of magnetic-nanoparticle formulations to desired targets in a patient, and more specifically, to systems and methods to better align magnetic forces with patient anatomy.

### SUMMARY

In various aspect, the present disclosure describes a method for configuring a medical cart, the method comprising: selecting a location of a headrest on the medical cart that is configured to support a patient in a first administration position and the second administration position; determining the location and position of a simulated patient, wherein the simulated patient is positioned in the first administration position and the second administration position; determining a plurality of target locations based on anatomy of the simulated patient; determining (X, Y, Z) coordinates of the plurality of target locations relative to an origin location at (0, 0, 0); selecting a position of the magnetic assembly, wherein the position is relative to the origin location, wherein the magnetic assembly comprises a plurality of magnetic elements, wherein each of the plurality of magnetic elements generates a magnetization that combines to produce a resulting magnetic field of the magnetic assembly; determining relative distance in space between the magnetic assembly and the plurality of target locations; selecting magnetic force directions relative to each of the plurality of target locations; determining a resulting magnetic field generated by the magnetic assembly such that the magnetic assembly generates the magnetic force directions relative to each of the plurality of target locations; determining a magnetization and direction for each of the plurality of magnetic elements; configuring the magnetic assembly where the plurality of magnetic elements are spatially located and directionally positioned such that the magnetic assembly produces magnetic force directions relative to each of the plurality of target locations.

### BRIEF DESCRIPTION OF THE DRAWINGS

The various aspects described herein, both as to organization and methods of operation, together with further objects and advantages thereof, may best be understood by reference to the following description, taken in conjunction with the accompanying drawings as follows.

FIG. 1 shows a cross-sectional view of a patient's head and the anatomy of the left ear and right ear, in accordance with at least one aspect of the present disclosure.

FIG. 2 shows a cross-sectional view of a patient in a first and second agent delivery position on a medical cart, in accordance with at least one aspect of the present disclosure.

FIG. 3 shows a system for administering an agent to a pediatric patient comprising a medical cart, in accordance with at least one aspect of the present disclosure.

FIGS. 4A and 4B illustrate a three dimension view and profile view of a magnetic assembly comprising a magnet of magnetic array, in accordance with at least one aspect of the present disclosure.

FIGS. 5A-5D shows a transparent alignment grid at the surface of the headrest, in accordance with at least one aspect of the present disclosure.

FIG. 6 shows a top view of the medical cart comprising safety bars or guard rails, in accordance with at least one aspect of the present disclosure.

FIG. 7 shows a profile view of the medical cart comprising a cantilever leg that may be folding up or down, in accordance with at least one aspect of the present disclosure.

FIG. 8 illustrates a system for determining magnetization and force direction for each magnetic element in a magnetic assembly, in accordance with at least one aspect of the present disclosure.

FIG. 9 illustrates a flow diagram for configuring of a medical cart, in accordance with at least one aspect of the present disclosure.

### DETAILED DESCRIPTION

The present disclosure describes a medical cart for treating medical conditions affecting the middle ears in patients, by using magnetic force to push the magnetic agent to a target position. In various aspects, a patient (e.g., a child or an adult) may lie or sit on the medical cart or bed and undergo a procedure of medical treatment with a magnetic agent. The magnetic agent comprises magnetic particles with attached therapies, such as drugs, proteins, or genes, and the magnetic agent is directed to the patient's middle ear or ears with magnetic forces, as disclosed in U.S. Patent Application Publication No. 2020/0146995, which is incorporated herein by reference in its entirety. In one aspect, the medical cart comprises a bed platform, a head rest, one or more guild rails, an alignment grid to guide alignment of the patient's head by a doctor or clinician, and other components to keep the patient comfortable (e.g. covering, cushions, etc). The medical cart further comprises a magnet assembly located beneath the bed platform, and is configured to pull magnetic particles into the middle ears of the patient. The magnetic array may be hidden by removable side coverings or fairings to provide a less intimidating appearance to the patient.

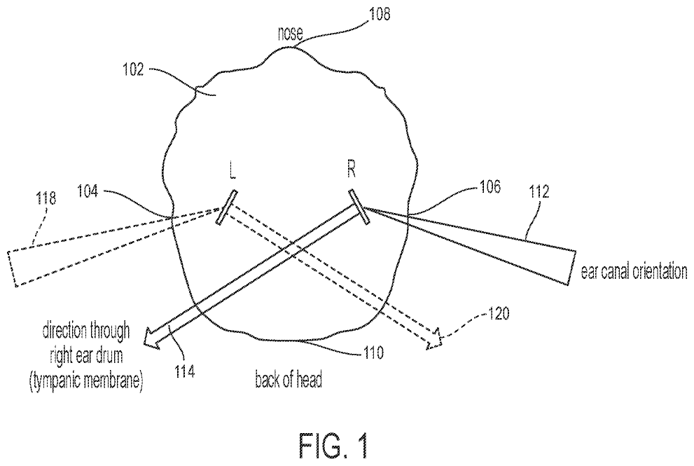

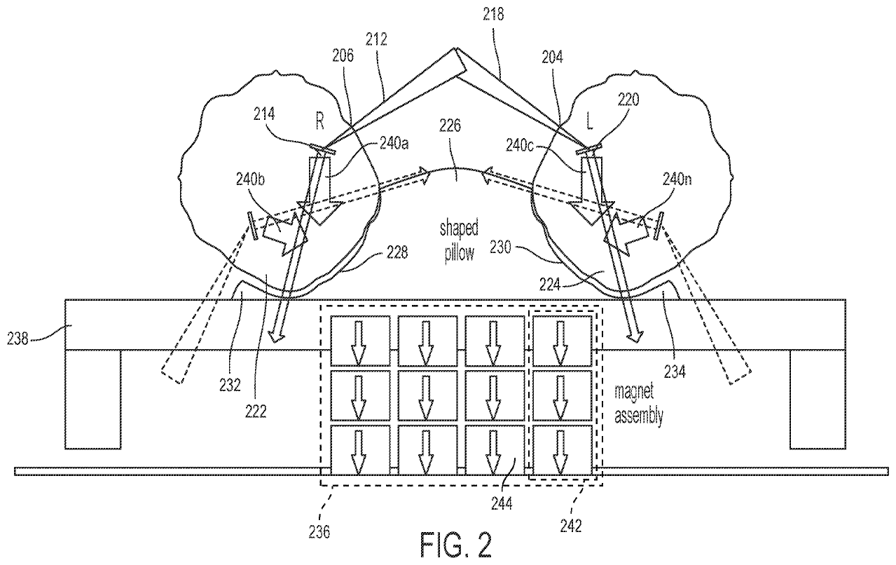

In various aspects, the medical cart is configured to treat one or both ears of the patient, and is optimized for the patient's conform during the treatment procedure. Additionally, the medical cart is designed for effective and easy use by the doctor or clinician, and can fit into a clinical setting (is safe and convenient to use, can be stowed away when not in use, etc.). In one aspect, the headrest comprises an alignment grid that guides a clinician to position the patient on the medical cart and aids in the administration of a magnetic therapeutic agent by the doctor or clinician. The shape of the headrest and the location of the alignment grids may be configured according to the location and proximity to the magnetic assembly, and in consideration of principles of physics (e.g. gravitational or magnetic force) and human anatomy constraints of the left and right ear, as shown in FIGS. 1 and 2.

FIG. 1 shows a cross-sectional view of a patient's head 102 and the anatomy of the left ear 104 and right ear 106. The cross-sectional view is a downward looking view from above the patient's head (transverse plane), with the nose 108 oriented in a top position and the back of the head 110 oriented in a bottom position. The orientation angle of the right ear canal 112 (outer ear) is shown by a conical marking. The orientation of the right ear drum 114 (right tympanic membrane) is marked by the arrow “direction through right ear drum”. The left ear canal orientation 118 and the left ear drum 120 are depicted with a dotted line. FIG. 1 illustrates an approximate or representative anatomical orientation of a patient, and it is understood that the exact orientations may vary from patient to patient.

These anatomical characteristics, illustrated by FIG. 1, are considered for determining the patient's optimal positioning for the administration of a therapeutic agent in the left and right ear. During the treatment procedure, the magnetic therapeutic agent is administered into the ear canal, through the outer ear, and is directed to the middle ear through a combination of gravitational force and magnetic force. The magnetic therapeutic agent comprises a plurality of therapeutic magnetic particles, and may be may be administered through the outer ear by a dropper or syringe, in a liquid composition or formulation.

FIG. 2 illustrates a cross-sectional view of a patient in a first and second agent administration position on a medical cart. The first position 222 is associated with administering the therapeutic agent in the patient's right ear and the second position 224 is associated with administering the therapeutic agent in the patient's left ear. The first position 222 and the second position 224 are optimal for a liquid formulation of the therapeutic agent to stay in the outer ear and not leak out due to gravity. The administration positions 222, 224 orient the respective outer ear canals in a position above the horizontal plane, such that the ear canals are not pointing at a downward angle. The headrest 226 shape comprises a first head contour 228 to support the patient's head in the first position 222 so that the patient can comfortably remain in the first position 222 while the magnetic and gravitation forces act upon the magnetic therapeutic agent and direct the agent to the middle ear. Similarly, the headrest 226 shape comprises a second head contour 230 for the patient to rest their head in the second position 224. The profile view of the headrest 226 resembles a normal distribution curve with wedges 232, 234 at the edges (e.g. first percentile and ninety-ninth percentile of a normal distribution) to secure the patient's head in place in the first position 222 and the second position 224. The headrest 226 may consist of a dense supportive material such as a foam or very firm memory foam.

FIG. 2 further shows a magnet assembly 236, located underneath the headrest 226, that produces a magnetic field and acts upon the magnetic therapeutic agent. In various aspects, the magnet assembly 236 may be covered by a removable fairing that prevents the magnet assembly 236 from being visible to the patient. Although middle ear conditions may affect any age group, middle ear infections are particularly prevalent in younger children. For a young child, it is desirable to have the treatment system look familiar, such as a simple bed, without showing any other features to the patient that may be intimidating to the parent or child. The removable fairings are configured to hide the magnetic assembly and make the medical cart appear to look like a traditional bed. This can help reduce patient stress and put the patient at ease. When the patient is calm, there is reduced motion and reduced likelihood that the patient's ear will drop below the horizontal plane. This ultimately allows the patient to remain in the optimal delivery position for a longer period of time and prevents the therapeutic agent from inadvertently dripping out of the administered ear. Thus, as shown in FIG. 2, the magnet array 236 is located underneath the bed platform 238 and positioned under the headrest 226, such that the magnetic array 236 is not visible to the patient.

FIG. 2 further illustrates a directional magnetic force produced by the magnetic array 236. The magnetic forces 240a-n are shown by arrows which also indicate the direction of the magnetic force. The location, size, shape, and magnetization of the magnet assembly 236 are selected so that the magnetic forces 240a, 240c points substantially through the patient's ear drum (tympanic membrane) 214, 220. For example the magnetic force 240a points through the right ear drum 214, while the right ear canal orientation 212 remains above the horizontal plane.

In various aspects, both right and left ears may be treated in a single administration session. In order to treat both ears in a single session, it is necessary that once a first ear is treated, that treating the second ear does not reverse or partially reverse the treatment just completed for the first ear. For example, a patient's right ear 206 is treated first in the first treatment position 222, allowing the magnetic therapeutic agent to be pulled to the patient's right middle ear space. The patient's left ear may then be subsequently treated after the right ear. While treating the left ear 204 in the second treatment position 224, it is necessary that “stray” magnetic force does not pull the magnetic particles out of the right ear 206. The first 222 and second treatment positions 224 are specifically designed so that magnetic forces 240b and 240n keep the magnetic therapeutic agent in the previously treated ear. For example, when the patient is in the second treatment positon 224, the magnetic force 240n on the right ear 206′ is oriented substantially through the right ear drum, but the magnetic force 240c on the left ear 204 is also still oriented through the left ear drum and is not oriented in such a way as to pull particles back out of the left middle ear space.

It is further desirable that the medical cart is configured to naturally support the patient's head, so that the head is oriented according to the magnetic force produced by the magnetic assembly. Thus, the system discloses a predetermined headrest shapes that specifically positon the patient's head according to gravitational and magnetic forces, for the first and second ear administration. Further, a transparent alignment grid may be visible or engraved into the headrest to aid the clinician in quickly and accurately placing the patient's head in the administration position.

In various aspects, the shape of the headrest, the location of the first and second headrest contours, and the location of alignment grid are determined according to the relative position to the magnetic assembly and the forces produced by the magnetic assembly. Accordingly, the magnetic assembly is configured according to the location, size, shape, and magnetization of magnets, to produce a desired magnetic force (e.g. 1 tesla of force experienced at the ear canal of the administration ear) on the therapeutic agent in patient's ear canals. The magnetic force, which is not the same nor equal to the magnetic field, is configured to produce directional force (vectors) for the left and right ear, in the first administration position 222 and the second administration positon 224.

If the vector magnetic field is denoted by B, where

B=[Bₓ(x, y, z), B_{y}(x, y, z), B_{z}(x, y, z)] is a 3-dimensional vector that varies across space X=(x, y, z), then the force on magnetic nanoparticles is given by

$$F = k [ \frac{d B}{d X} ]$$

B and is itself also a 3-dimensional vector that varies across space, i.e. F=[Fₓ(x, y, z), F_{y},(x, y, z), F_{z}(x, y, z)]. Here,

$$\frac{d B}{d X}$$

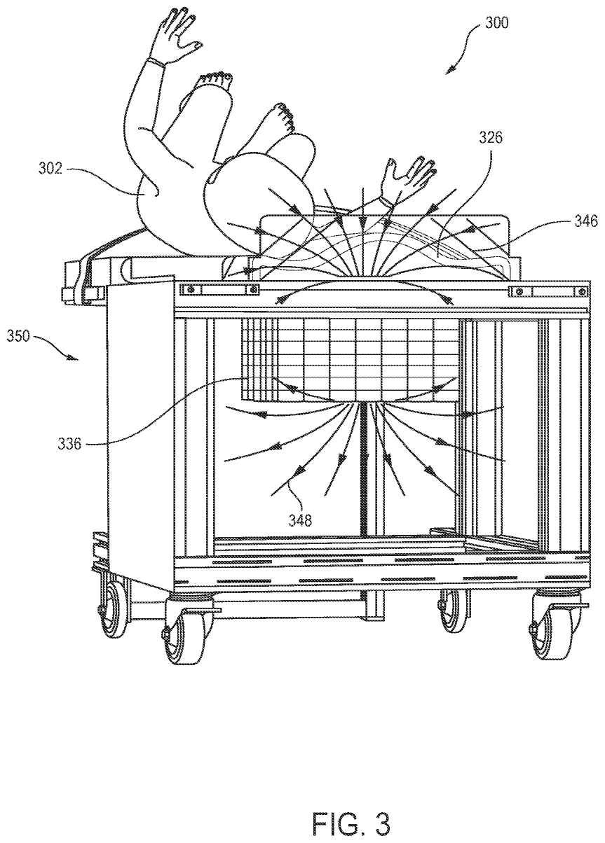

is the Jacobian matrix, and k is a coefficient whose properties depend based on the size and material parameters of the magnetic1 nanoparticles. Alternatively and equivalently, the magnetic force F can also be written as

$$F = \frac{1}{2} grad ( {‖ B ‖}^{2} )$$

where grad is the gradient, ∥B∥ is the norm of B. We disclose that the selection of the magnet assembly was chosen so that the magnetic force F=[Fₓ(x, y, z), F_{y} (x, y, z), F_{z}(x, y, z)] points in the desired directions, meaning it matches the directions shown in FIG. 3. This selection was done by conducting a mathematical optimization over magnet element placement, to find possible magnet placements that create forces in the right directions, for intended patient head orientations, for both ears.

FIG. 3 shows a system 300 for administering a magnetic therapeutic agent to a pediatric patient 302 comprising a medical cart 350. In the present aspect, the magnetic therapeutic agent composition comprises a magnetic nanoparticle particle, such as iron, attached to therapeutic component that can be administered to the patient. The system is configured to push or pull the magnetic particles of the agents into the patient's middle ears by the magnetic force that is a result of the magnetic field 348 produced by the magnetic assembly 336. Thus the therapeutic is pushed or pulled into the target location. In one aspect, the system 300 can be configured for patients 302 as young as 6 months old. The headrest 326 for the patient's head may be selected or configured based on the patient's head diameter. The magnetic assembly 336 is shown, in a cut-away view, under the headrest, and may not be visible to the patient or others around the patient.

In one aspect, the headrest can be shaped as shown, and the magnet is under it inside the cart. The patient's natural head position may be set by the headrest, and can orient the ears so that the magnetic force points from ear canal through the ear drum into the middle ear. The medical cart is configured to treat one ear at a time, without “untreating” the previously treated ear with the magnet assembly.

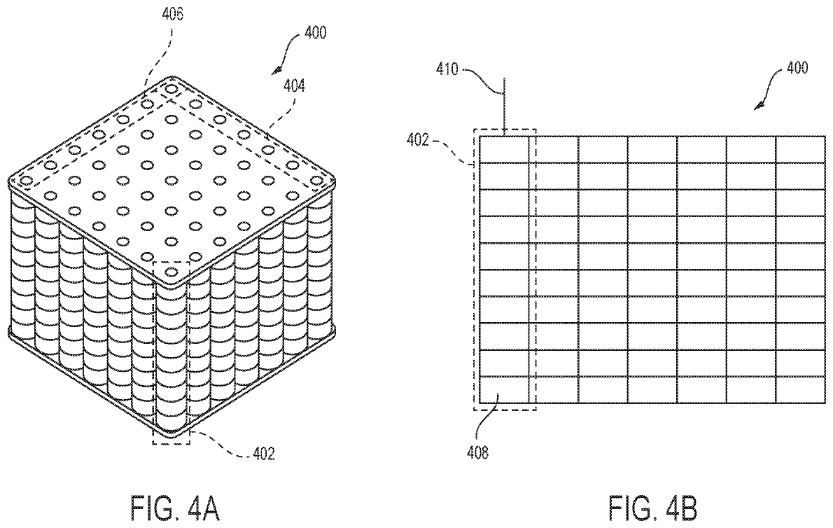

FIGS. 4A-4B illustrate a three-dimension view and profile-view of a rectangular magnetic assembly 400 comprising a magnetic array. The magnetic assembly 400 is configured to provide a predetermined magnetic force on the magnetic nanoparticles so that the therapeutic agent is directed across the ear drum. Additionally, that patient is positioned such that the direction of the magnetic forces are oriented in the same direction as both ear drums, which is different and distinct from the direction of the magnetic field. Accordingly, the magnetic field produced by the magnetic assembly must be designed for both head orientations; the first position 222 and second position 224 as illustrated in FIG. 2. Finally, the magnetic assembly may be designed and configured based on cost, ease, and safety of manufacturing.

In various aspects, the magnetic assembly 400 may comprise electromagnets, permanent magnets, or an array of permanent magnets. FIGS. 4A-4B illustrate and aspect of the magnetic assembly comprising of multiple strong but smaller permanent magnets, rather than a single larger and stronger magnet. Smaller magnets can be sourced more easily than one large one, and can be handled more safely during the manufacturing process. The magnetic assembly 400 of FIGS. 4A-4B comprises a magnetic array of a plurality of pillars 402 that are arranged in according to 7 rows 404 and 7 columns 406. Additionally, the magnetic array may also be configured in an analogous N rows×M columns structure, such that the N×M structure provides a predetermined amount of magnetic force on the magnetic nanoparticles to sufficient to of the magnetic agent to across the ear drum. In one aspect, the magnet assembly is configured to produce 1 tesla of force on a magnetic therapeutic agent in the ear canal of an administration ear.

In one aspect, the pillars 402 of the magnet assembly 400 may comprises a plurality of small disc magnets 408, such as Neodymium N42 disc magnets where each disc is 2 inches in diameter by 1 inch in height, with a ¼ inch hole in the middle for a supporting rod to go through. The plurality of disc magnets may be bonded together with a supporting rod 410, where each of the plurality of discs may be magnetized in the same vertical direction with opposite polarity ends attracted to each other. In one aspect, there may be 10 magnetic discs 408 for each pillar 402 in the array. In one aspect, the magnet assembly may be approximately 14 inches wide, +/−1 inch, 14 inches deep+/−1 inch, and 10 inches tall+/−1 inch.

The magnetic fields of the plurality of discs combine to create a resulting magnetic field for each pillar. Similarly, the magnetic fields of the plurality of pillars combine to create a resulting magnetic field for the magnetic assembly. Therefore, the same resulting magnetic fields may be produced by different magnetic assembly pillar and disc combinations. As discussed above, the resulting magnetic field is determined according to a calculated magnetic force on the magnetic nanoparticles.

The magnetic fields of the plurality of discs combine to create a resulting magnetic field for each pillar. Similarly, the magnetic fields of the plurality of pillars combine to create a resulting magnetic field for the magnetic assembly. Therefore, the same resulting magnetic fields may be produced by different magnetic assembly pillar and disc combinations. As discussed above, the resulting magnetic field is determined according to a calculated magnetic force on the magnetic nanoparticles.

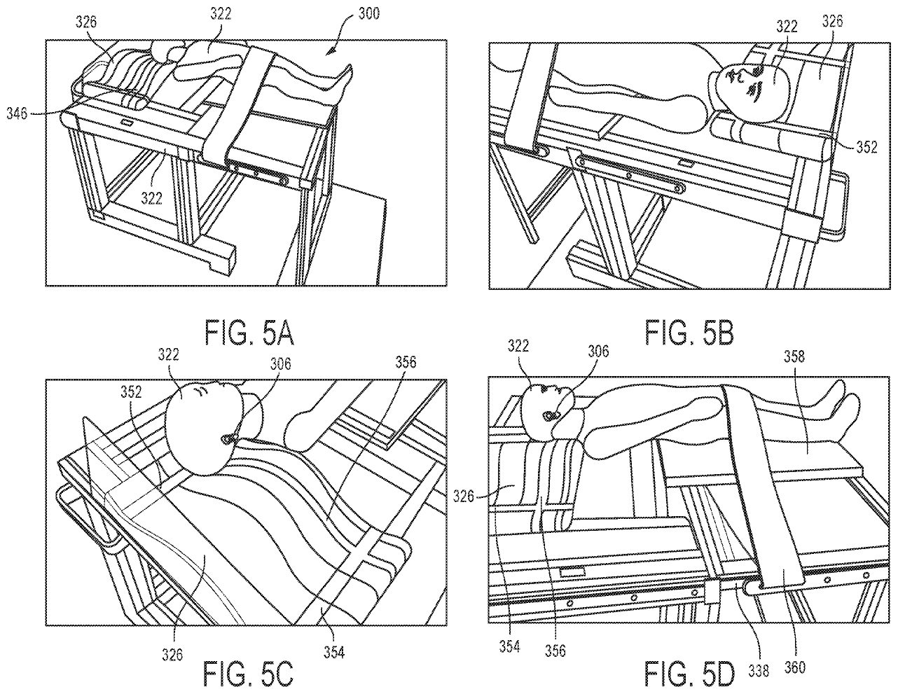

FIGS. 5A-D show a medical simulation mannequin positioned according to a transparent alignment grid 346 on the surface of a headrest 325. The alignment grid 346 allows the clinician to easily align the patient's head according to the magnetic forces, invisible to the naked eye, produced by the magnetic assembly. The alignment grid may be a light color indicator in contrast with a dark color of the headrest so that the alignment grid is shown in high contrast and can be seen through a protective covering. A protective or sanitary covering may be used in conjunction with the alignment grid and allows the clinician to view the alignment grid through the protective covering, without the patient making direct contact with the headrest. In another aspect, the alignment grid 346 may be engraved into the surface of the headrest 326. Similarly, the physical engraving may be perceived through a protective covering.

The headrest 326 may also be removable and would require one or more headrest markings to affix or align the headrest so that the alignment grid correctly aligns with the magnet assembly beneath the upper bed platform 338. It is anticipated that the clinician would stand behind or to the side of the patient and help align the patient's head with the alignment grid 346 of the headrest 326. Additionally, the headrests 326 may be different sizes for different patient head diameters and may require alignment grids to be uniquely calibrated for the shape of the headrest. In one aspect, the shape and size of the first and second head contours of the headrest are different sizes to meeting smaller or larger patients. Accordingly, the alignment grid is calibrated so that the patient's head is in the desired position for the magnetic force to act upon the magnetic therapeutic agent.

FIG. 5C shows the medical simulation mannequin in the first administration position 322 with the right ear 306 positioned for treatment. The alignment grid 346 further comprises a vertical first position indicator 352, vertical second position indicator 354, and a horizontal position indicator 356. In one aspect, the clinician aligns the patient's eyes according to the horizontal position indicator 356 and the bridge of their nose according to the vertical first position indicator 352. Additionally, the clinician aligns the patient's head so that the outer right ear 306 is above horizontal.

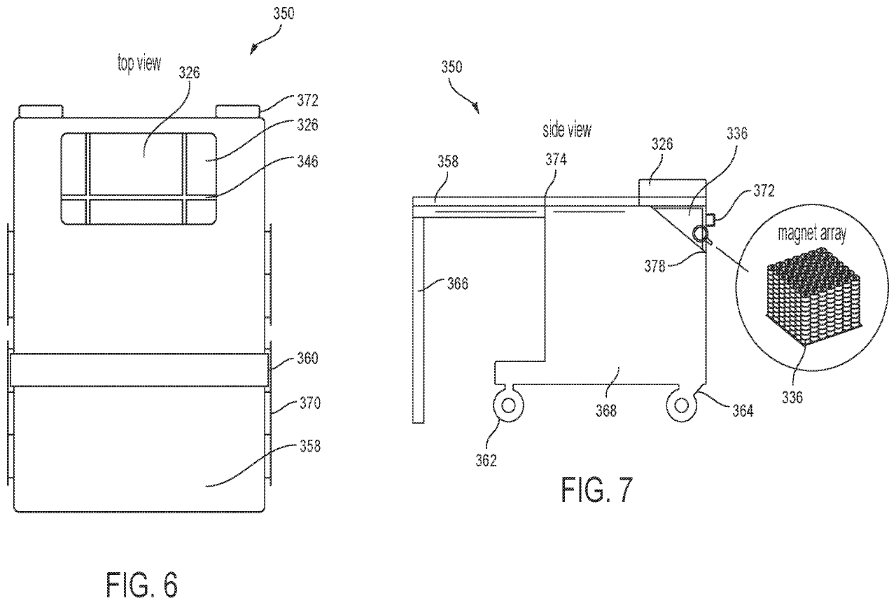

FIG. 5D shows the medical cart optionally comprising a mattress 358 positioned on top of the upper bed platform 338, an adjustable security belt 360, and guard rails 370 to help keep the patient in place. FIG. 6 further shows a top view of the medical cart 350 comprising safety bars or guard rails 370. While the patient may be secured by the adjustable security belt 360, it is desirable to further support the sides of the medical cart 350 with guard rails 370. The medical cart is configured to patient to be moved from a first position to a second position and the guard rails may prevent the patient from falling of the cart during a position transition.

FIG. 7 shows a profile view of the medical cart 350 comprising a cantilever leg 366 that may be folding up or down. In one aspect, the medical cart 350 may be a compact design so that it may fit into an existing exam room without requiring dedicated storage space. The medical cart 350 may comprises a folding hinge 374 on the upper bed platform 338 to fold to a smaller storage size. Additionally, the medical cart 350 may further comprise one or more casters 362 for positioning and relocation of the medical cart 350. At least one of the casters 362 may be lockable with respect to transverse motion along the ground to allow the medical cart 350 to pivot about a pivoting axis perpendicular to the ground. When position is selected, that medical cart 350 is secured in place by the cantilever leg 366 and one or more caster brakes 364. Thus after use, the cart can be folded up and rolled into a storage area. FIG. 7 further shows the removable side fairings 368 covering the magnet assembly 336 from the field of view from the patient. The magnet assembly 336 is located at the proximal end 378 of the cart and is aligned in the center of the longitudinal axis of the cart.

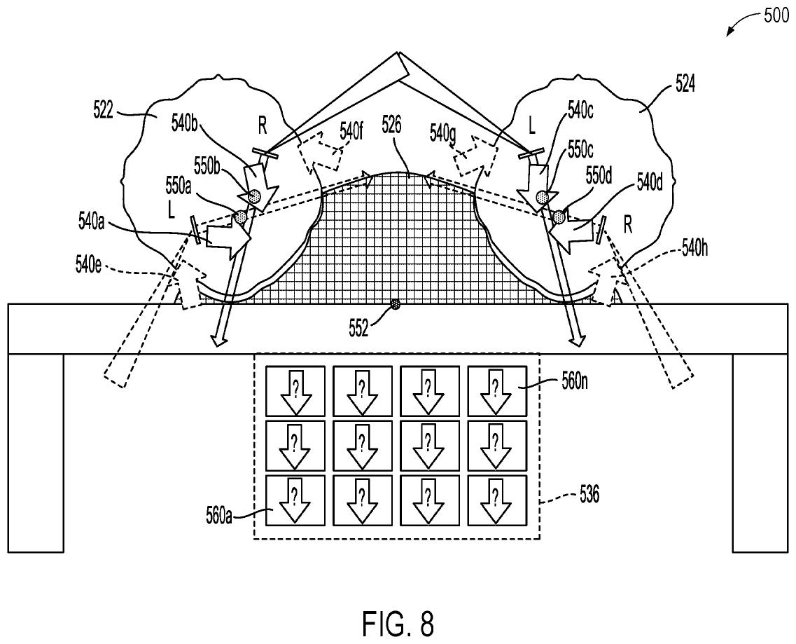

FIG. 8 illustrates a system 500 for determining magnetization and force directions for each magnetic element in a magnetic assembly 536. The magnetic assembly 536 may be configured to generate magnetic forces 540a-d that create pushing and/or pulling force on a magnetic therapeutic agent at specific locations 550a-d relative to a simulated patient 522, 524 on the medical cart. In one aspect, the simulated patient 522, 524 may be a child between the ages of 4-6 years old. The simulated patient 522, 524 may be modeled such that the ear canal is simulated from the median ear canal structure for patients between a predetermined age range and development. Additionally, the location of the simulated patient's head is in a 3D space relative to the medical cart and is based on a median head size for a patient between the predetermined age range and development.

The simulated patient's head orientation is determined according to the headrest position on the medical cart, alignment grid 346 on the headrest 526, and the patient's head position in the first administration position 522 and second administration position 524. Once the location and orientation of the simulated patient is known, various target locations relative to the patient's ear canal can be determined.

For example, when the patient is in the first administration position, it may be desirable to generate a pull force at the middle ear of the active administration ear to pull a therapeutic agent. Simultaneously, it may be desirable to generate a push force at the outside ear of the passive administration ear to offset the force of gravity and prevent a therapeutic agent from dipping out of a previously treated ear. In another example, a push force and pull force may be generated at along the same ear canal.

FIG. 8 further illustrates a system 500 for configuring a plurality of magnetic elements 560a-n within a magnetic assembly 536 to achieve predetermined magnetic force and direction 540a-g at target locations 550a-d, relative to a patient's ear canal. The magnetization and orientation of each magnetic element 560a-n in the magnetic assembly 536 is determined based on a quadratic mapping. In various aspects, the magnetization for each magnetic element 560a-n in the magnetic assembly 536 may be configured to produce the maximum magnetic saturation or maximum magnetic moment per unit volume for the magnetic element material. In one example, high permeability iron alloys have a maximum magnetic saturation of 1.6-2.2 T.

A plurality of target locations is determined based on the need for the therapeutic agent to reach the middle ear and to stay at the middle ear while treating the other ear. The specific location of target locations 550a-d are determined in three dimensional space based on (X,Y, Z) coordinates relative to the medical cart. In one aspect, the origin location 552, at coordinates (0, 0, 0), is on the medical at the midpoint of the headrest in the length and width direction. Target locations 550a-d are determined based on their relative locations to the origin 552.

The magnetic force and direction 540a-d is generated at each target locations 550a-d based on the configuration of the plurality of magnetic elements 560a-n that make up the magnetic assembly 536. In another aspect the plurality of magnetic elements 560a-n may be configured based on magnetic force and direction 540a-h. The plurality of magnetic elements 560a-n generates individual magnetic fields that combine to create a resulting magnetic field of the magnetic assembly 536. In various aspects, the quadratic mapping may be used to determine a magnetic assembly configuration.

In one aspect, the quadratic mapping is m′ Qₖm, where m is a vector that represents the to-be-selected magnetization at each magnetic elements 560a-n of the magnetic assembly 536. Where, m=[m₁x,m₁y,m₁z; m₂x,m₂y,m₂z; . . . , m_{N}x,m_{N}y,m_{N}z], and the triplet (mⱼx, mⱼy, mⱼz) is the x, y, z magnetization of the j^{th} component. By selecting different (mⱼx, mⱼy, mⱼz), the magnetization can be selected in any direction for that j^{th} component, where j^{th} component is one magnetic elements 560a-n in the magnetic assembly 536. The matrix, Qₖ, encapsulates the resulting direction of the magnetic force on an agent at location Rₖ=(Xₖ, Yₖ, Zₖ). In various aspects, Rₖ is a target location, such as the location of the center of the right ear drum when the right ear is being treated, or target locations 550a-d in FIG. 8. Based on the position, orientation, and location of the simulated patient's head, a set of desired force directions can be selected, such as 540a-d in FIG. 8. Meaning, the desired force directions, v, of the quadratic mapping, m′ Qₖm, are:
m′ Q₁m=v₁→direction 1=d₁
m′ Q₂m=v₂→direction 2=d₂
m′ Q₃m=v₃→direction 3=d₃
m′Q_{K}m=v_{K}→direction K=d_{K}

Overall, there is a complex proportional relationship between the magnetization directions m generated by the magnetic assembly 536 and the force directions, v=[v₁, v₂, . . . , v_{K}], exerted at the target locations R=[R₁, R₂, . . . , Rₖ]. The quadratic mapping equations, shown above, may be used to select a magnetization, m, to achieve a force directions, v, to approach the desired directions d=[d₁, d₂, . . . , d_{K}] as closely as possible. The purpose of configuring the magnetic elements 560a-n of the magnetic assembly 536 is to create a magnetic system that orients forces in space to match the simulated patient anatomy, as illustrated in FIG. 8.

In various aspects, the magnetic assembly may generate push nodes, pull nodes, or complex directional forces are neither “pushing” nor “pulling” based on the arrangement and position of the magnetic elements in the magnetic assembly. In one example, forces may be arranged in the desired directions based on the target locations. Additionally, a directional force may be substantially oriented at an angle through the patient's ear drum (tympanic membrane).

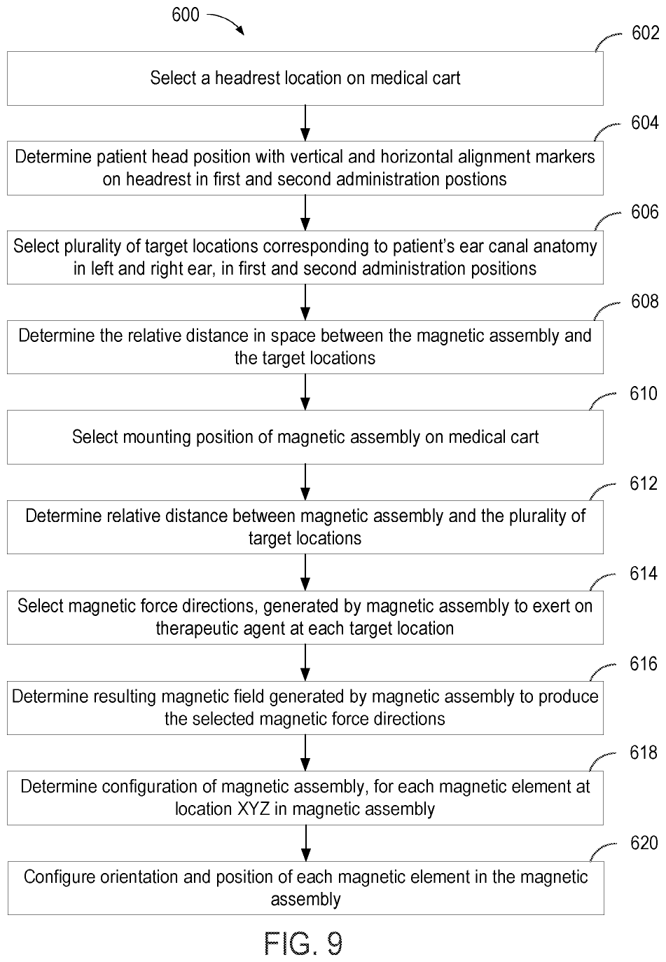

FIG. 9 illustrates a flow diagram 600 for configuring of a medical cart. The configurator selects 602 a first fixed position of a headrest on a medical cart. The configurator determines 604 a simulated patient position on the medical cart in the first and second administration positons. The simulated patient position is determined based on a media head size and anatomical characteristics for a patient a predetermined age or development. The configurator selects 506 a plurality of target location based on the patient's ear canal location in space in the first administration position and second administration position. Four target locations may be selected corresponding to both ears in the first and second administration position. When the patient is in the first administration position, a first target location may correspond to the active administration ear, where the doctor administers the therapeutic agent, and a second target location may correspond to the passive administration ear, where the doctor has already administered the therapeutic agent to this ear. When the patient is in the second administration position, a third target location may correspond to the active administration ear, where the doctor administers the therapeutic agent, and a fourth target location may correspond to the passive administration ear, where the doctor has already administered the therapeutic agent to this ear. Although only three locations will typically be needed in practice, four locations are used regardless of whether the doctor starts with a right ear or left ear. Once the plurality of target locations are selected, the relative (X, Y, Z) coordinates of the target locations is determined 608 in space, relative to the origin location (0,0,0). The configurator selects 610 a mounting location for the magnetic assembly on the medical cart, relative to the origin location (0,0,0). The configurator determines 612 a relative distance in space between the target locations and the magnetic assembly. The configurator selects 614 a plurality of magnetic force directions relative to the target locations. The configurator determines 616 a resulting magnetic field generated by the magnetic assembly, such that the magnetic field is sufficient to produce the selected force directions relative to the target locations. The configurator determines 618 a magnetization and direction for each of the magnetic elements in the magnetic assembly, where each magnetization and direction is determined based on the (X,Y,Z) location of the magnetic element in the magnetic assembly. The configurator configures 620 the magnetic assembly such that each magnetic element is configured according to the determined magnetization and direction.

Various additional aspects of the subject matter described herein are set out in the following numbered examples:

Example 1: A method for configuring a medical cart, the method comprising:
selecting a location of a headrest on the medical cart that is configured to support a patient in a first administration position and the second administration position; determining the location and position of a simulated patient, wherein the simulated patient is positioned in the first administration position and the second administration position; determining a plurality of target locations based on anatomy of the simulated patient; determining (X, Y, Z) coordinates of the plurality of target locations relative to an origin location at (0, 0, 0); selecting a position of the magnetic assembly, wherein the position is relative to the origin location, wherein the magnetic assembly comprises a plurality of magnetic elements, wherein each of the plurality of magnetic elements generates a magnetization that combines to produce a resulting magnetic field of the magnetic assembly; determining relative distance in space between the magnetic assembly and the plurality of target locations; selecting magnetic force directions relative to each of the plurality of target locations; determining a resulting magnetic field generated by the magnetic assembly such that the magnetic assembly generates the magnetic force directions relative to each of the plurality of target locations; determining a magnetization and direction for each of the plurality of magnetic elements; configuring the magnetic assembly where the plurality of magnetic elements are spatially located and directionally positioned such that the magnetic assembly produces magnetic force directions relative to each of the plurality of target locations.

Example 2: The method of Example 1, wherein determining the plurality of target locations is further comprising: determining a first target locations relative to the simulated patient in the first administration position, wherein the first target location is based on ear canal anatomy of a first ear of the simulated patient in an active administration ear; determining a second target locations relative to the simulated patient in the first administration position, wherein the second target location is based on ear canal anatomy of a second ear of the simulated patient in a passive administration ear; determining a third target locations relative to the simulated patient in the second administration position, wherein the third target location is based on ear canal anatomy of the second ear of the simulated patient in the active administration ear; and determining a fourth target locations relative to the simulated patient in the second administration position, wherein the fourth target location is based on ear canal anatomy of the first ear of the simulated patient in the passive administration ear.

Example 3: The method of Example 2, wherein the magnetization of each of the plurality of magnetic elements is the maximum magnetic saturation based on the composition material of the plurality of magnetic elements.

Example 4: The method of Examples 3, wherein the material composition of the plurality of magnets is same material composition for all of the plurality of magnets.

Example 5: The method of Examples 1-4, wherein each magnetic elements of the plurality of magnetic elements are selectively positioned and placed at (X, Y, Z) coordinate locations relative to the origin location, within the magnetic assembly.

Example 6: The method of claim 2-5, wherein the resulting magnetic field creates directional forces at a desired angle or angles based on the plurality of target locations.

Example 7: The method of claim 6, wherein the desired angle or angles comprise orientation angles through ear drums of the patient.

Example 8: The method of Examples 2-5, wherein the resulting magnetic field creates a pushing force in the direction of the middle ear at the plurality of target locations.

Example 9: The method of Examples 2-5, wherein the resulting magnetic field creates a pushing force in the direction of the ear canal at the first and the third target location, and a pulling force in the direction of the ear canal at the second and the four target location, wherein the direction of the pushing force and pulling force is from the outside inward along the ear canal.

Example 10: The method of Examples 2-9, wherein the resulting magnetic field creates a pulling force in the direction of the ear canal at the first and the third target location, and a push node direction of the ear canal at the second and the four target location, wherein the direction of the pushing force and pulling force is from the outside inward along the ear canal.

Example 11: The method of Example 1-10, wherein determining each of the plurality of target locations is based the alignment of simulated patient with a vertical position indicator and a horizontal position indicator on an alignment grid of the headrest.

Example 12: The method of Example 2-11, the first target location and the second target location is the middle ear of the first ear, and the third target location and the fourth target location is the middle ear of the second ear.

Example 13: The method of Examples 1-12, wherein the first target location is the middle ear of the first ear and the third target location is the middle ear of the first ear.

Example 14: The method of Examples 1-13, the second target location is the outer ear of the first ear and the forth target location is the outer ear of the second ear.

Example 15: The method of Examples 1-14, wherein the ear canal anatomy of the simulated patient is modeled on a predetermined age range and development of the patient.

Example 16: The method of Examples 1-15, wherein the simulated patient age range and development is a median head size and median ear canal of a child between the ages of 3-7 years old.

Example 17: The method of Examples 1-16, wherein the magnetization and direction for each of the plurality of magnetic elements is determined according mⱼ=(mⱼx, mⱼy, mⱼz), where m is the magnetization and j is the j^{th} magnetic element in the magnetic assembly.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a control circuit computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December, 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1. A method for configuring a medical cart, the method comprising:
selecting a location of a headrest on the medical cart that is configured to support a patient in a first administration position and a second administration position;
determining the location and position of a simulated patient, wherein the simulated patient is positioned in the first administration position and the second administration position;
determining a plurality of target locations based on anatomy of the simulated patient; determining (X, Y, Z) coordinates of the plurality of target locations relative to an origin location at (0, 0, 0);
selecting a position of a magnetic assembly, wherein the position is relative to the origin location, wherein the magnetic assembly comprises a plurality of magnetic elements, wherein each of the plurality of magnetic elements generates a magnetization that combines to produce a resulting magnetic field of the magnetic assembly;
determining relative distance in space between the magnetic assembly and the plurality of target locations;
selecting magnetic force directions relative to each of the plurality of target locations;
determining a resulting magnetic field generated by the magnetic assembly such that the magnetic assembly generates the magnetic force directions relative to each of the plurality of target locations;
determining a magnetization and direction for each of the plurality of magnetic elements; and
configuring the magnetic assembly where the plurality of magnetic elements are spatially located and directionally positioned such that the magnetic assembly produces magnetic force directions relative to each of the plurality of target locations.

2. The method of claim 1, wherein determining the plurality of target locations is further comprising:
determining a first target locations relative to the simulated patient in the first administration position, wherein the first target location is based on ear canal anatomy of a first ear of the simulated patient in an active administration ear;
determining a second target locations relative to the simulated patient in the first administration position, wherein the second target location is based on ear canal anatomy of a second ear of the simulated patient in a passive administration ear;
determining a third target locations relative to the simulated patient in the second administration position, wherein the third target location is based on ear canal anatomy of the second ear of the simulated patient in the active administration ear; and
determining a fourth target locations relative to the simulated patient in the second administration position, wherein the fourth target location is based on ear canal anatomy of the first ear of the simulated patient in the passive administration ear.

3. The method of claim 2, wherein the magnetization of each of the plurality of magnetic elements is the maximum magnetic saturation based on the composition material of the plurality of magnetic elements.

4. The method of claim 3, wherein the material composition of the plurality of magnets is same material composition for all of the plurality of magnets.

5. The method of claim 1, wherein each magnetic elements of the plurality of magnetic elements are selectively positioned and placed at (X, Y, Z) coordinate locations relative to the origin location, within the magnetic assembly.

6. The method of claim 2, wherein the resulting magnetic field creates directional forces at a desired angle or angles based on the plurality of target locations.

7. The method of claim 6, wherein the desired angle or angles comprise orientation angles through ear drums of the patient.

8. The method of claim 2, wherein the resulting magnetic field creates a pushing force in the direction of the middle ear at the plurality of target locations.

9. The method of claim 2, wherein the resulting magnetic field creates a pushing force in the direction of the ear canal at the first and the third target location, and a pulling force in the direction of the ear canal at the second and the fourth target location, wherein the direction of the pushing force and pulling force is from the outside inward along the ear canal.

10. The method of claim 2, wherein the resulting magnetic field creates a pulling force in the direction of the ear canal at the first and the third target location, and a push node direction of the ear canal at the second and the fourth target location, wherein the direction of the pushing force and pulling force is from the outside inward along the ear canal.

11. The method of claim 1, wherein determining each of the plurality of target locations is based the alignment of simulated patient with a vertical position indicator and a horizontal position indicator on an alignment grid of the headrest.

12. The method of claim 2, wherein the first target location and the second target location is the middle ear of the first ear, and the third target location and the fourth target location is the middle ear of the second ear.

13. The method of claim 1, wherein the first target location is the middle ear of the first ear and the third target location is the middle ear of the first ear.

14. The method of claim 1, wherein the second target location is the outer ear of the first ear and the fourth target location is the outer ear of the second ear.

15. The method of claim 1, wherein the ear canal anatomy of the simulated patient is modeled on a predetermined age range and development of the patient.

16. The method of claim 15, wherein the simulated patient age range and development is a median head size and median ear canal of a child between the ages of 3-7 years old.

17. The method of claim 1, wherein the magnetization and direction of each of the plurality of magnetic elements is determined according mⱼ=(mⱼx, mⱼy, mⱼz), where m is the magnetization and j is the j^{th} magnetic element in the magnetic assembly.
